(19) United States
(12) Patent Application Publication
(10) Pub. No.: US 2004/0122524 A1
(43) Pub. Date: Jun. 24, 2004

(54) Bi-polar hip prosthetic devices employing diffusion-hardened surfaces

(75) Inventors: Gordon Hunter, Memphis, TN (US); Jeffrey E. Schryver, Cordova, TN (US); Steve Miller, Germantown, TN (US)

Correspondence Address: SMITH & NEPHEW, INC., 1450 E. BROOKS ROAD, MEMPHIS, TN, 38116, US

(73) Assignee: Smith & Nephew, Inc., Memphis, TN (US)

(21) Appl. No.: 10/323,039

(22) Filed: Dec. 18, 2002

(51) Int. Cl. A61F 2/36

(52) U.S. Cl. 623/22.18; 623/23.11; 623/23.53

(57) ABSTRACT

An orthopedic implant having diffusion-hardened surfaces employed at inner and outer load-bearing surfaces. Preferably, the orthopedic implant is a bipolar hip prosthetic device and system where a coating of oxidized zirconium is formed at the articulating, load-bearing surface of the acetabular component and at the articulating, load-bearing surface of the femoral head. The acetabular component has a polymeric cup, made from a bio-compatible material, such as UHMWPE.

### BACKGROUND OF THE INVENTION

1. Field of the Invention

The field of this invention relates generally to orthopedic prosthetic devices, and more particularly to bipolar hip prosthetic devices employing diffusion-hardened surfaces.

2. General Background of the Invention

The excellent corrosion resistance of zirconium has been known for many years. Zirconium displays excellent corrosion resistance in many aqueous and non-aqueous media and for this reason has seen an increased use in the chemical process industry and in medical applications. A limitation to the wide application of zirconium in these areas is its relatively low resistance to abrasion and its tendency to gall. This relatively low resistance to abrasion and the tendency to gall is also demonstrated in zirconium alloys.

U.S. Pat. No. 2,987,352 to Watson first disclosed a method of producing zirconium bearings with a specific form of oxidized zirconium as a surface layer. The specific form of oxidized zirconium is a blue-black or blue oxidized zirconium. The method of Watson was refined by Haygarth (U.S. Pat. No. 4,671,824) resulting in improved abrasion resistance and better dimensional control of the oxidized product. U.S. Pat. No. 5,037,438 to Davidson first demonstrated the many advantages that are realized through the use of the specific form of oxidized zirconium on zirconium and zirconium alloy substrates in prosthetic devices. Davidson extended this work to include surfaces subject to nitridation in U.S. Pat. No. 5,180,394. Other U.S. Patents of Davidson (U.S. Pat. Nos. 5,152,794; 5,370,694; 5,372,660; 5,415,704; 5,496,359; and 5,549,667) demonstrate the use of this specific form of zirconium oxide or zirconium nitride in other prosthetic application. All of the aforementioned patents of Davidson are incorporated by reference as though fully disclosed herein. The advantages of these surfaces include increased strength, low friction and high wear resistance. U.S. Pat. Nos. 5,037,438 and 5,180,394 to Davidson, respectively, disclose a method of producing zirconium alloy prostheses with an oxidized zirconium surface and a nitrided zirconium surface. The work of Watson and Davidson teach a specific form of oxidized or nitrided zirconium which possesses all of the advantages of ceramic materials while maintaining the strength of metallic surfaces. While the present invention is not intended to be limited by theory, the oxide or nitride layer are believed to be characterized by the presence of free oxygen or nitrogen which diffuses into the interior of the material, near the metallic substrate. The resulting “diffusion hardened” surfaces have oxide or nitride layers that possess properties that combine the unique advantages of both ceramic and metal surface, while simultaneously minimizing the disadvantages of these materials. All of the U.S. Patents cited above to Davidson, Watson, and Haygarth are incorporated by reference as though fully set forth herein. While the early work of Davidson focused on pure zirconium and alloys of zirconium in which zirconium was the predominant metal, later work has shown that this is not necessary in order to form the desired diffusion hardened oxide. For instance, an alloy of 74 wt % titanium, 13 wt % niobium and 13 wt % zirconium (“Ti-13-13”) will form the diffusion hardened oxidation layer used herein. Ti-13-13 is taught in U.S. Pat. No. 5,169,597 to Davidson et al. By effectively taking advantage of the unique properties of such diffusion-hardened layers on prosthetic devices, the useful service life of the device is greatly improved. The improvement was realized by improving the wear resistance of the contacting surfaces of an implant (most notably the articulating surfaces), thereby lengthening the useful service life of the implant.

U.S. Pat. No. 5,037,438 and U.S. Pat. No. 5,180,394 to Davidson recognized that a thin coating of zirconium oxide, nitride, carbide or carbonitride is especially useful on the portions of prosthetics, especially metallic orthopedic implants for load-bearing surfaces which are subject to high rates of wear. An example cited is a unipolar design hip-system prothesis, where a femoral head of the hip-system prosthesis engages a single counter-bearing surface in a conventional acetabular component affixed to the pelvis. The acetabular component has a cup often made of a softer material such as ultra-high molecular weight polyethylene. This example illustrates only a single load-bearing interface. The Davidson '438 and '394 patents further recognized that zirconium oxide and nitride coatings on non-load bearing surfaces of an orthopedic implant that contact tissue provides a barrier between the metallic prosthesis and body tissue which prevents the release of metal ions and corrosion of the implant.

The zirconium oxide or nitride coating provides the prosthesis with a thin, dense, low friction, wear resistant, bio-compatible surface ideally suited for use on articulating surfaces of joint prostheses wherein a surface or surfaces of the joint articulates, translates or rotates against mating joint surfaces. The zirconium oxide or nitride may be employed on the articulating surfaces of femoral and tibial (meniscal bearing) surfaces of knee joints.

Another Davidson patent, U.S. Pat. No. 5,415,704 further discusses the creation of a diffusion-hardened surface of bio-compatible metallic metals and alloys, suitable for use as material for a medical implant, including in particular, niobium, titanium, and zirconium based alloys. The '704 patent discusses various methods of oxidizing or nitriding metals and alloys to provide a fine oxide or nitride dispersion.

The Davidson patents, however, did not address the issue of a bipolar hip system having a diffusion-hardened surface disposed on an articulating, outer load-bearing acetabular component and an articulating, inner load-bearing femoral head component. The bipolar hip raises new concerns where a prosthetic device has multiple articulating load-bearing surfaces. With the bipolar hip prosthetic, a first articulating load-bearing surface exists at the acetabular component/acetabulum interface. A second articulating load-bearing surface exists at the femoral head component/polymeric cup interface. The Davidson patents only addressed single load-bearing articulating joint surfaces having a zirconium oxide surface where the load-bearing joint surface either articulated against body tissue or against another load-bearing joint surface.

The compound bearing surfaces of bipolar hip prosthetic provides greater overall range of motion than either unipolar designs or conventional total hip arthroplasty. It has been found, however, that a common wear problem exists at the acetabular component/acetabulum interface and the femoral head component/polymeric cup interface. Due to the articulating of the acetabular component, wear occurs at both the interfaces.

The outer surface of the acetabular component articulates with the tissue surfaces, such as cartilege or bone. Friction occurs at this interface, not only, causing wear of the outer surface of the acetabular component, but also upon the tissue where the outer surface articulates. Wear against the tissue especially occurs if the outer surface of the acetabular component incurs marring. Additionally, wear occurs at the femoral head interface with the polymeric cup.

Therefore, a need exists for a prosthetic implant that provides a strengthened, low friction, highly wear resistant surface at compound bearing surfaces of the implant. In particular, to provide reduced wear and improved strength, it is desireable to have a bipolar hip with a diffusion hardened surface on the first load-bearing, articulating surface and a diffusion hardened surface on the second load-bearing, articulating surface.

### SUMMARY OF THE INVENTION

In one aspect of the invention a prosthetic device comprises an acetabular component, a polymeric, bio-compatible material lining the interior surface of the acetabular component, and a femoral head component rotatably secured in the polymeric material. The acetabular has an outer surface and an interior surface. The acetabular component is formed of zirconium, hafnium, niobium, tantalum or alloys thereof. The said femoral head component is formed of zirconium, hafnium, niobium, tantalum or alloys thereof. A first diffusion-hardened coating is formed on at least a part of the outer surface of the femoral head component. Also, a second diffusion-hardened coating is formed on at least a part of said femoral head component.

In one embodiment the polymeric insert may articulate against the inner surface of the acetabular component. The surface of inner surface of the acetabular component may be highly polished to provide a low wear surface on which the acetabular component may articulate. In another embodiment, the polymeric insert is securely fixed within the acetabular component and so that the polymeric insert does not articulate against the inner surface of the acetabular component.

In one emobodiment, the diffusion-hardened coating of the acetabular component or the femoral head component may employ a thin coating of blue-black or black zirconium oxide. The diffusion-hardened surfaces also may be a thin coating of oxidized metal selected from one or more metals from the group consisting of hafnium, zirconium, niobium and tantalum.

In a specific embodiment, the diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof.

In another embodiment the acetabular component or the femoral head component is formed of zirconium or zirconium alloy where the diffusion-hardened coating comprises blue-black or black oxidized zirconium.

In another embodiment, the first or second diffusion-hardened coating is a thin coating of blue-black or black zirconium oxide.

In one embodiment of the invention the polymeric, bio-compatible material is UHMWPE (ultra-high molecular-weight polyethylene).

In one aspect of the invention the thickness of the first diffusion-hardened coating is about the same thickness of the second diffusion-hardened coating.

In another embodiment, a third diffusion-hardened coating is formed on at least a part of the inner surface of the acetabular component. The third diffusion-hardened coating may be a diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof. Also, in one embodiment, the third diffusion-hardened coating is formed of zirconium or zirconium alloy and said diffusion-hardened coating comprises blue-black or black oxidized zirconium.

In an alternative embodiment, the femoral head component is formed of zirconium, hafnium, niobium, tantalum, or alloys thereof. The femoral head component may have a diffusion-hardened oxide or nitride coating, and that coating may be oxidized zirconium, oxidized hafnium, oxidized niobium, oxidized tantalum, nitrided zirconium, nitrided hafnium, nitrided niobium, nitrided tantalum or combinations thereof.

In a preferred embodiment, the femoral head component is comprised of zirconium or zirconium alloy and the diffusion-hardened oxide coating is made of blue-black or black oxidized zirconium.

In another embodiment of the present invention, there is an acetabular component formed of alloy having a composition comprising from about 10 to about 20 wt % niobium or from about 35 to about 50 wt % niobium; from about 13 to about 20 wt % zirconium; and the balance titanium; a diffusion-hardened oxide or nitride coating on at least a part of the load bearing surface of the acetabular component.

In yet another embodiment of the present invention, there is a femoral head component formed of alloy having a composition comprising from about 10 to about 20 wt % niobium or from about 35 to about 50 wt % niobium; from about 13 to about 20 wt % zirconium; and the balance titanium; a diffusion-hardened oxide or nitride coating on at least a part of the load-bearing surface of the femoral head component.

In one embodiment, the femoral head component has a composition consisting essentially of about 74 wt % titanium, about 13 wt % niobium, and about 13 wt % zirconium.

In another embodiment, the acetabular component has a composition consisting essentially of about 74 wt % titanium, about 13 wt % niobium, and about 13 wt % zirconium.

In one embodiment of the invention, a prosthesis for implantation in a patient includes a prosthesis body with a first component, a second component, and a biocompatible, polymeric liner, for example UHMWPE, disposed between said first and second component. The first component has an outer diffusion-hardened, load-bearing surface. This first component is sized and shaped to engage and articulate within a cavity of the patient. For example, the cavity may be the acetabulum of the femur, the glenoid cavity of the scapula, or a cavity formed by a physician for the purpose of articulation with the prosthetic. The second component also has a diffusion-hardened, load-bearing surface. The second component however is sized and shaped to engage or cooperate with the interior surface of the polymeric liner. The second component is preferably rotatably secured within the polymeric liner.

### BRIEF DESCRIPTION OF THE DRAWINGS

A better understanding of the invention can be obtained from the detailed description of exemplary embodiments set forth below, when considered in conjunction with the appended drawings, in which:

FIG. 1 is a schematic illustrating an embodiment of the present invention as a bipolar hip prosthetic device;

FIG. 2 is a schematic illustrating a cross-sectional view of FIG. 1; and

FIG. 3 is a schematic illustrating an embodiment of the present invention attached to a femoral stem.

### DETAILED DESCRIPTION OF EXEMPLARY EMBODIMENTS

As used herein, “a” or “an” may mean one or more. As used herein in the claim(s), when used in conjunction with the word “comprising”, the words “a” or “an” may mean one or more than one. As used herein, “another” may mean at least a second or more.

As used herein, as it refers to interacting surfaces on a prosthetic device, the term “cooperate” is defined as any type of interaction, including an articulating interaction, a non-articulating interaction, and any and all intermediate levels of interaction.

As used herein, “diffusion-hardened surface” is defined as a type of abrasion resistant surface formed by certain specific in-situ oxidation or nitridation processes. The surface is characterized by being oxidized or nitrided relative to the substrate upon which it is situated. It is oxidized or nitrided by an in-situ oxidation or nitridation process by which oxygen or nitrogen diffuses from the surface toward the interior substrate domain. Specific examples of the oxidation or nitridation processes are provided herein. When used in reference to the underlying substrate material, it is synonymous with “surface hardened”. Also synonymously, the surface oxide or nitride layer is also referred to as “diffusion-bonded”. An oxidized or nitrided zirconium surface, as those terms are used herein, are examples of a diffusion hardened surface; other metals or metal alloys may also form diffusion-hardened surfaces by oxidation or nitridation. In all discussions herein referring to various applications and embodiments of diffusion-hardened surfaces on prosthetic devices, it should be understood that discussions with respect to oxidized surfaces apply equally to nitrided surfaces.

As used herein, “metallic” may be a pure metal or an alloy.

As used herein, “nitridation” is defined as the chemical process by which a substrate material, preferably a metal is combined with nitrogen to form the corresponding nitride.

As used herein, “zirconium alloy” is defined as any metal alloy containing zirconium in any amount greater than about 10% by weight of zirconium. Thus, an alloy in which zirconium is a minor constituent at about 10% by weight or greater is considered a “zirconium alloy” herein. Similarly, a “metal alloy” of any other named metal (e.g., a hafnium alloy or a niobium alloy; in these cases, the named metal is hafnium and niobium, respectively) is defined as any alloy containing the named metal in any amount greater than about 10% by weight.

The invention provides a bipolar hip prosthetic device with a diffusion-hardened surface disposed on an articulating, outer-bearing acetabular component. The invention provides a novel prosthetic implant that provides a strengthened, low friction, highly wear resistant surface at compound bearing surfaces of the implant. In particular, the invention relates to a bipolar hip with a diffusion hardened surface on the first load-bearing, articulating surface and a diffusion hardened surface on a second load-bearing, articulating surface.

The diffusion-hardened surface of either component is preferably a thin coating of blue-black or black zirconium oxide. Additionally, the diffusion-hardened surface of either component may be a thin coating of oxidized metal selected from one or more metals from the group consisting of hafnium, zirconium, niobium and tantalum. The thickness of the diffusion-hardened surface of the first component is about the same thickness of the diffusion-hardened surface of the second component.

In another embodiment of the invention, a prosthesis for implantation in a patient, includes a bipolar hip prosthetic device. The bipolar hip prosthetic device includes an acetabular component, a polymeric insert, and a femoral head component.

The acetabular component is configured to be surgically implanted into a patient's acetabulum. The acetabular component, generally spherical in shape, employs an outer diffusion-hardened, load-bearing surface. The acetabular component houses a polymeric, bio-compatible insert, for example UHMWPE, that lines the interior surface of the acetabular component. This lining forms a cup for the femoral head. The interior surface of the acetabular component may be hardened or not. The interior surface of the acetabular component is preferably diffusion-hardened because the interior surface is subject to micro-motion or fretting, however, the interior surface is a non-articular surface and hardening is not mandatory. The interior surface of the polymeric lining is preferably spherically shaped for articulation of the femoral head.

In one embodiment the polymeric insert may articulate against the inner surface of the acetabular component. The surface of inner surface of the acetabular component may be highly polished to provide a low wear surface on which the acetabular component may articulate. In another embodiment, the polymeric insert is securely fixed within the acetabular component and so that the polymeric insert does not articulate against the inner surface of the acetabular component.

The femoral component is configured to be rotatably secured within the cup of the polymeric insert. The femoral head, generally spherical in shape, employs an outer diffusion-hardened, load-bearing surface for articulation with the polymeric insert.

The invention provides orthopedic implants having diffusion-hardened oxide or nitride surfaces such as oxidized zirconium or nitrided zirconium. More generally, metals or metal alloys of titanium, vanadium, niobium, hafnium and/or tantalum may be used as substrate materials to form suitable diffusion-hardened oxide surface layers. Most of the examples herein deal with zirconium or zirconium alloy substrates and surface layers of oxidized zirconium or nitrided zirconium; however, it should be understood that other metals such as hafnium, vanadium, titanium, niobium, tantalum, and their alloys, are amenable to the present invention. In order to form continuous and useful oxide or nitride coatings over the desired surface of the metal alloy prosthesis substrate, the metal alloy should preferably contain from about 80 to about 100 wt. % of the desired metal, and more preferably from about 95 to about 100 wt. %. It should be noted that in some cases, lower amount of the desired metal are possible. In some cases, alloys where the desired metal is at about 10% by weight or greater may yield acceptable results. For example, an alloy of about 74 wt % titanium, about 13 wt % niobium and about 13 wt % zirconium (“Ti-13-13”) can be successfully used herein. Ti-13-13 is taught in U.S. Pat. No. 5,169,597 to Davidson et al. Thus, while levels of the desired metal of about 10% by weight or greater are known to produce acceptable results, increasing this level continuously gives progressively better results, with at least 80% by weight, and at least 95% by weight, being the preferred and most preferred levels, respectively.

In the case of either oxidized or nitrided zirconium, oxygen, niobium, and titanium, among others, may be included as common alloying elements in the alloy with often times the presence of hafnium. Yttrium may also be alloyed with the zirconium to enhance the formation of a tougher, yttria-stabilized zirconium oxide coating during the oxidation of the alloy. While oxidized or nitrided zirconium is used for illustrative purposes herein, it should be understood that the teachings apply analogously to the other possible metal candidates as well. While such zirconium containing alloys may be custom formulated by conventional methods known in the art of metallurgy, a number of suitable alloys are commercially available. In the case of oxidized zirconium some commercial alloys include, among others Zircadyne 705, Zircadyne 702, and Zircalloy.

The base metal and metal alloys are cast or machined by conventional methods to the shape and size desired to obtain a suitable prosthesis substrate. The substrate is then subjected to process conditions which cause the in situ formation of a tightly adhered, diffusion-bonded coating of zirconium oxide or zirconium nitride on its surface. The term “diffusion-hardened” and “diffusion-bonded” are used in reference to the desired oxides or nitrides because the formation of these particular surfaces is characterized by the diffusion of oxygen or nitrogen from the surface towards the interior (i.e., approaching the unoxidized substrate, native metal or metal alloy). It is believed that this diffusion of oxygen or nitrogen is what imparts the high strength and high wear resistance to these surfaces. The process conditions for formation include, for instance, air, steam, or water oxidation or oxidation in a salt bath. These processes ideally provide a thin, hard, dense, low friction, wear-resistant zirconium nitride or blue-black or black wear-resistant zirconium oxide film or coating of thicknesses typically on the order of several microns (10⁻⁶ meter) on the surface of the prosthesis substrate. Below this coating, diffused oxygen or nitrogen from the oxidation or nitridation process increases the hardness and strength of the underlying substrate metal.

The air, steam and water oxidation processes are described for zirconium and zirconium alloys in now-expired U.S. Pat. No. 2,987,352 to Watson, the teachings of which are incorporated by reference as though fully set forth. These methods may also be applied to metals and alloys of hafnium, titanium, vanadium, niobium, and tantalum. In the case of zirconium or zirconium alloy, the air oxidation process provides a firmly adherent black or blue-black layer of zirconium oxide of highly oriented monoclinic crystalline form. If the oxidation process is continued to excess, the coating will whiten and separate from the metal substrate. The oxidation step may be conducted in either air, steam or hot water. For convenience, the metal prosthesis substrate may be placed in a furnace having an oxygen-containing atmosphere (such as air) and typically heated at 700° F.-1100° F. up to about 6 hours. However, other combinations of temperature and time are possible. When higher temperatures are employed, the oxidation time should be reduced to avoid the formation of the white oxide.

The oxide layer should range in thickness from about 1 to about 20 microns; however, a range of from about 1 to about 5 microns is preferred. The overall average thickness can be controlled by the parameters of time and temperature. For example, furnace air oxidation at 1000° F. for 3 hours will form an oxide coating on Zircadyne 705 about 2-3 microns thick, oxidation at 1175° F. for 1 hour results in an overall average oxide coating of about 4-5 microns thick, and oxidation at 1175° F. for 3 hours results in an overall average oxide coating of about 10-11 microns thick. As additional examples, one hour at 1300° F. will form an oxide coating about 14 microns in thickness, while 21 hours at 1000° F. will form an oxide coating thickness of about 9 microns. Using different combinations of oxidation times and higher oxidation temperatures will increase or decrease this thickness, but higher temperatures and longer oxidation times may compromise coating integrity, depending upon the nature of the substrate and other factors. For thicker coatings of oxide, some trial and error may be necessary. Of course, because in the usual case only a thin oxide is necessary on the surface, only very small dimensional changes, typically less than 10 microns over the thickness of the prosthesis, will result. In general, thinner coatings (1-5 microns) have better attachment strength.

One of the salt-bath methods that may be used to apply the oxide coatings to the metal alloy prosthesis, is the method of U.S. Pat. No. 4,671,824 to Haygarth, the teachings of which are incorporated by reference as though fully set forth. In the case of oxidized zirconium, the salt-bath method provides a similar, slightly more abrasion resistant blue-black or black zirconium oxide coating. The method requires the presence of an oxidation compound capable of oxidizing zirconium in a molten salt bath. The molten salts include chlorides, nitrates, cyanides, and the like. The oxidation compound, sodium carbonate, is present in small quantities, up to about 5 wt %. The addition of sodium carbonate lowers the melting point of the salt. As in air oxidation, the rate of oxidation is proportional to the temperature of the molten salt bath and the '824 patent prefers the range 550° C.-800° C. (1022° F.-1470° F.). However, the lower oxygen levels in the bath produce thinner coatings than for furnace air oxidation at the same time and temperature. A salt bath treatment at 1290° F. for four hours produces an oxide coating thickness of roughly 7 microns.

Whether air oxidation in a furnace or salt bath oxidation is used, the oxide coatings are quite similar in hardness. For example, if the surface of a wrought Zircadyne 705 (Zr, 2-3 wt. % Nb) prosthesis substrate is oxidized, the hardness of the surface shows a dramatic increase over the 200 Knoop hardness of the original metal surface. The surface hardness of the resulting blue-black zirconium oxide surface following oxidation of Zircadyne 705 by either the salt bath or air oxidation process is approximately 1700-2000 Knoop hardness.

In the case of nitridation of zirconium and zirconium alloys, an analogous procedure is used. As in the oxide case, the nitride layer should range in thickness from about 1 to about 20 microns; however, a range of from about 1 to about 5 microns is preferred. Even though air contains about four times as much nitrogen as oxygen, when zirconium or a zirconium alloy is heated in air as described above, the oxide coating is formed in preference to the nitride coating. This is because the thermodynamic equilibrium favors oxidation over nitridation under these conditions. Thus, to form a nitride coating the equilibrium must be forced into favoring the nitride reaction. This is readily achieved by elimination of oxygen and using a nitrogen or ammonia atmosphere instead of air or oxygen when a gaseous environment (analogous to “air oxidation”) is used. In order to form a zirconium nitride coating of about 5 microns in thickness, the zirconium or zirconium alloy prosthesis should be heated to about 800° C. for about one hour in a nitrogen atmosphere. Thus, apart from the removal of oxygen (or the reduction in oxygen partial pressure), or increasing the temperature, conditions for forming the zirconium nitride coating do not differ significantly from those needed to form the blue-black or black zirconium oxide coating. Any needed adjustment would be readily apparent to one of ordinary skill in the art.

When a salt bath method is used to produce a nitride coating, then the oxygen-donor salts should be replaced with nitrogen-donor salts, such as, for instance cyanide salts. Upon such substitution, a nitride coating may be obtained under similar conditions to those needed for obtaining an oxide coating. Such modifications as are necessary, may be readily determined by those of ordinary skill in the art. Alternatively, the zirconium nitride may be deposited onto the zirconium or zirconium alloy surface via standard physical or chemical vapor deposition methods, including those using an ion-assisted deposition method. It is preferred that the physical or chemical vapor deposition methods be carried out in an oxygen-free environment. Techniques for producing such an environment are known in the art, for instance the bulk of the oxygen may be removed by evacuation of the chamber and the residual oxygen may be removed with an oxygen scavenger.

When the zirconium or zirconium alloy is provided with a zirconium porous bead, zirconium wire mesh surface, or textured surface, then this surface layer can also be coated with zirconium oxide or nitride, as the case may be, to provide protection against metal ionization in the body.

These diffusion-bonded, low friction, highly wear resistant oxidized or nitrided zirconium coatings are grown in-situ and used on the surfaces of orthopedic implants subject to conditions of wear. Such surfaces include, but are not limited to, the articulating surfaces of knee joints, elbows and hip joints. As mentioned before, in the case of hip joints, the femoral head and stem are typically fabricated of metal alloys while the acetabular cup may be fabricated from ceramics, metals or organic polymer-lined metals or ceramics. However, the acetabular cup may be fabricated of a metal or metal alloy that forms a diffusion-hardened surface.

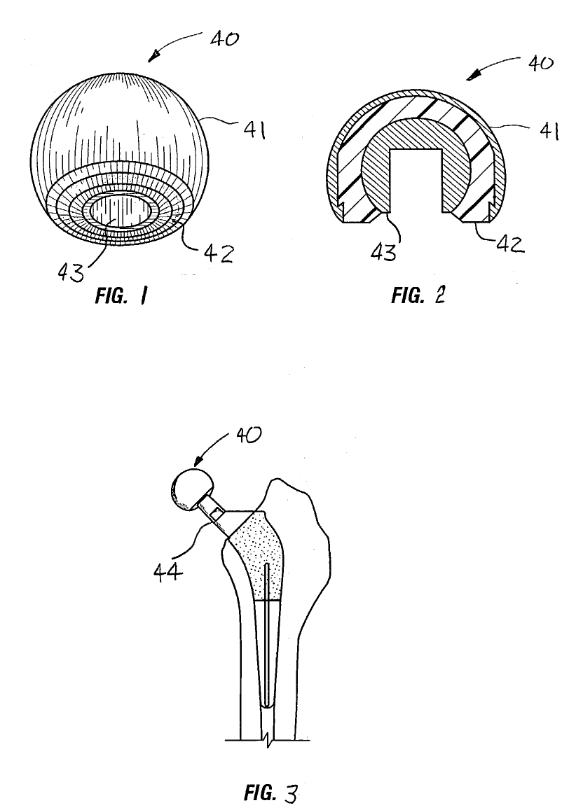

FIG. 1 shows an embodiment of the present invention designated generally by the numeral 40. In FIG. 3, a bipolar-hip prosthetic system is shown including an acetabular component 41, a polymeric insert 42, and a femoral head component 43. A polymeric insert 42 is inserted into the acetabular component 41. The polymeric insert generally does not articulate against the inside surface of the acetabular componenet 41. The femoral head component 43 is rotably secured within the polymeric insert 42.

When implanted, the acetabular component 41 articulates within the acetabulum. The acetabular component 41, generally spherical in shape, employs an outer diffusion-hardened, load-bearing surface for articulation with bodily tissue. The femoral component 43, generally spherical in shape, employs an outer diffusion-hardened, load-bearing surface for articulation with the polymeric insert 42.

The diffusion-hardened surface of the acetabular component 41 or the femoral component 43 may employ a thin coating of blue-black or black zirconium oxide. The diffusion-hardened surfaces may also consist of a thin coating of oxidized metal selected from one or more metals from the group consisting of hafnium, zirconium, niobium and tantalum.

Referring now to FIG. 2, a cross-section of the bipolar-hip prosthetic of FIG. 1 is shown. As better illustrated in FIG. 2, the femoral component 43 is shown positioned within the polymeric insert 42. As previously discussed, this femoral component 43 has a diffusion-hardened surface that articulates with the polymeric insert.

Lastly referring to FIG. 3, an embodiment of the bipolar-hip prosthetic system is illustrated. The bipolar-hip prosthetic 40 is attached to a femoral stem 44 which is shown implanted in the femur. Two load-bearing, articulating surfaces provide better range of motion than the unipolar-hip prosthetic.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the invention described in the specification. As one of ordinary skill in the art will readily appreciate from the disclosure of the present invention, devices, means, metals and alloys existing or later to be developed that perform substantially the same function or achieve substantially the same result as the corresponding embodiments described herein may be utilized according to the present invention. Accordingly, the appended claims are intended to include within their scope such devices, means, and metals and alloys.

| | | |
|---|---|---|
| 2,987,352 | June 1961 | Watson |
| 4,671,824 | June 1987 | Haygarth |
| 5,037,438 | August 1991 | Davidson |
| 5,152,794 | October 1992 | Davidson |
| 5,169,597 | December 1992 | Davidson et al. |
| 5,180,394 | January 1993 | Davidson |
| 5,370,694 | December 1994 | Davidson |
| 5,372,660 | December 1994 | Davidson et al. |
| 5,415,704 | May 1993 | Davidson |
| 5,496,359 | March 1996 | Davidson |
| 5,549,667 | August 1996 | Davidson |

What is claimed is:

1. A bipolar hip prosthetic device comprising:
a) an acetabular component, the acetabular having an outer surface and an interior surface, said acetabular component formed of zirconium, hafnium, niobium, tantalum or alloys thereof;
b) a polymeric, bio-compatible material lining the interior surface; and
c) a femoral head component, the femoral head component being rotatably secured in said polymeric, bio-compatible material, said femoral head component formed of zirconium, hafnium, niobium, tantalum or alloys thereof;
d) a first diffusion-hardened coating on at least a part of the outer surface of the femoral head component; and
e) a second diffusion-hardened coating on at least a part of said femoral head component.

2. The bipolar hip prosthetic device of claim 1 wherein said first diffusion-hardened coating is an oxide coating selected from the group consisting of oxidized zirconium, oxidized hafnium, oxidized niobium, oxidized tantalum and combinations thereof.

3. The bipolar hip prosthetic device of claim 1 wherein said first diffusion-hardened coating is a nitride coating selected from the group consisting of nitrided zirconium, nitrided hafnium, nitrided niobium, nitrided tantalum and combinations thereof.

4. The bipolar hip prosthetic device of claim 1 wherein said second diffusion-hardened coating is an oxide coating selected from the group consisting of oxidized zirconium, oxidized hafnium, oxidized niobium, oxidized tantalum and combinations thereof.

5. The bipolar hip prosthetic device of claim 1 wherein said second diffusion-hardened coating is a nitride coating selected from the group consisting of nitrided zirconium, nitrided hafnium, nitrided niobium, nitrided tantalum and combinations thereof.

6. The bipolar hip prosthetic device of claim 1 wherein said acetabular component is formed of zirconium or zirconium alloy and said diffusion-hardened coating comprises blue-black or black oxidized zirconium.

7. The bipolar hip prosthetic device of claim 1 wherein said femoral head component is formed of zirconium or zirconium alloy and said diffusion-hardened coating comprises blue-black or black oxidized zirconium.

8. The prosthetic system of claim 1, wherein the first diffusion-hardened coating is a thin coating of blue-black or black zirconium oxide.

9. The prosthetic system of claim 1, wherein the second diffusion-hardened coating is a thin coating of blue-black or black zirconium oxide.

10. The prosthetic system of claim 1, wherein the polymeric bio-compatible material is UHMWPE.

11. The prosthesis of claim 1, wherein the thickness of the first diffusion-hardened coatng is about the same thickness of the second diffusion-hardened coating.

12. The prosthesis of claim 1, further comprising a third diffusion-hardened coating on at least a part of the inner surface of the acetabular component.

13. The bipolar hip prosthetic device of claim 12 wherein said third diffusion-hardened coating is an oxide coating selected from the group consisting of oxidized zirconium, oxidized hafnium, oxidized niobium, oxidized tantalum and combinations thereof.

14. The bipolar hip prosthetic device of claim 12 wherein said third diffusion-hardened coating is a nitride coating selected from the group consisting of nitrided zirconium, nitrided hafnium, nitrided niobium, nitrided tantalum and combinations thereof.

15. The bipolar hip prosthetic device of claim 12 wherein said third diffusion-hardened coating is formed of zirconium or zirconium alloy and said diffusion-hardened coating comprises blue-black or black oxidized zirconium.

16. The bipolar hip prosthetic device of claim 12, wherein said third diffusion-hardened coating is a thin coating of blue-black or black zirconium oxide.

17. The bipolar hip prosthetic device of claim 1, wherein the femoral head component has a composition consisting essentially of about 74 wt % titanium, about 13 wt % niobium, and about 13 wt % zirconium.

18. The bipolar hip prosthetic device of claim 1, wherein the acetabular component has a composition consisting essentially of about 74 wt % titanium, about 13 wt % niobium, and about 13 wt % zirconium.

19. The bipolar hip prosthetic device of claim 1, wherein the femoral head component is formed of alloy having a composition comprising from about 10 to about 20 wt % niobium or from about 35 to about 50 wt % niobium; from about 13 to about 20 wt % zirconium; and the balance titanium.

20. The bipolar hip prosthetic device of claim 1, wherein the acetabular component is formed of alloy having a composition comprising from about 10 to about 20 wt % niobium or from about 35 to about 50 wt % niobium; from about 13 to about 20 wt % zirconium; and the balance titanium.

21. The bipolar hip prosthetic device of claim 1, wherein the polymeric material is configured to articulate against the inner surface of the acetabular component.

22. The bipolar hip prosthetic device of claim 21, wherein the inner surface of the acetabular component is highly polished.

23. A prosthetic device, comprising:
(a) a first component being sized and shaped to engage or cooperate with a cavity of a patient, the first component having a diffusion-hardened coating on at least a part of the outer surface of the first component, the first component formed of zirconium, hafnium, niobium, tantalum or alloys thereof;
(b) a biocompatible, polymeric liner disposed within said first component; and
(c) a second component having a diffusion-hardened coating on at least a part of the outer surface of the second component, the second component being sized and shaped to engage or cooperate with the interior surface of the polymeric liner, the first component having a diffusion-hardened coating on at least a part of the outer surface, the first component formed of zirconium, hafnium, niobium, tantalum or alloys thereof.

24. The prosthetic device of claim 23 wherein said diffusion-hardened coating of the first component is an oxide coating selected from the group consisting of oxidized zirconium, oxidized hafnium, oxidized niobium, oxidized tantalum and combinations thereof.

25. The prosthetic device of claim 23 wherein said diffusion-hardened coating of the first component is a nitride coating selected from the group consisting of nitrided zirconium, nitrided hafnium, nitrided niobium, nitrided tantalum and combinations thereof.

26. The prosthetic device of claim 23 wherein said diffusion-hardened coating of the second component is an oxide coating selected from the group consisting of oxidized zirconium, oxidized hafnium, oxidized niobium, oxidized tantalum and combinations thereof.

27. The prosthetic device of claim 23 wherein said diffusion-hardened coating of the second component is a nitride coating selected from the group consisting of nitrided zirconium, nitrided hafnium, nitrided niobium, nitrided tantalum and combinations thereof.

28. The prosthetic device of claim 23 wherein said first component is formed of zirconium or zirconium alloy and said diffusion-hardened coating comprises blue-black or black oxidized zirconium.

29. The prosthetic device of claim 23 wherein said second component is formed of zirconium or zirconium alloy and said diffusion-hardened coating comprises blue-black or black oxidized zirconium.

30. The prosthetic device of claim 23, wherein the diffusion-hardened coating of the first component is a thin coating of blue-black or black zirconium oxide.

31. The prosthetic device of claim 23, wherein the diffusion-hardened coating of the second component is a thin coating of blue-black or black zirconium oxide.

32. The prosthetic device of claim 23, wherein the polymeric bio-compatible material is UIIMWPE.

33. The prosthetic device of claim 23, wherein the thickness of the diffusion-hardened coating of the first component is about the same thickness of the diffusion-hardened coating of the second component.

34. The prosthetic device of claim 23, further comprising a third diffusion-hardened coating on at least a part of an inner surface of the first component.

35. The prosthetic device of claim 34 wherein said third diffusion-hardened coating is an oxide coating selected from the group consisting of oxidized zirconium, oxidized hafnium, oxidized niobium, oxidized tantalum and combinations thereof.

36. The prosthetic device of claim 34 wherein said third diffusion-hardened coating is a nitride coating selected from the group consisting of nitrided zirconium, nitrided hafnium, nitrided niobium, nitrided tantalum and combinations thereof.

37. The prosthetic device of claim 34 wherein said third component is formed of zirconium or zirconium alloy and said diffusion-hardened coating comprises blue-black or black oxidized zirconium.

38. The prosthetic device of claim 34, wherein said third diffusion-hardened coating is a thin coating of blue-black or black zirconium oxide.

39. The prosthetic device of claim 23 wherein the first component is an acetabular head adapted to cooperate with and slide against cartilage tissue of a pelvis.

40. The prosthetic device of claim 23 wherein the first component is a head of a humeral implant adapted to cooperate with natural body tissue of a glenoid of a patient.

41. The prosthetic device of claim 23 wherein the first component is a glenoid prosthesis adapted to cooperate with natural tissue of a humerus.

42. The prosthetic device of claim 23 wherein the first component is a prosthetic with at least one condyle of a femoral component of a knee joint prosthesis adapted to cooperate against natural tissue of a tibia.

43. The prosthetic device of claim 17 wherein the first component is a tibial component of a knee joint prosthesis adapted to cooperate with natural tissue of condyles.
